(19) United States
(12) Patent Application Publication
(10) Pub. No.: US 2022/0395671 A1
(43) Pub. Date: Dec. 15, 2022

(54) VACUUM DRESSING FOR USE WITH GUIDE TUBE

(71) Applicant: Cardiac Assist Holdings, LLC, Ann Arbor, MI (US)

(72) Inventors: Allen B. Kantrowitz, Ann Arbor, MI (US); Barry N. Gellman, Ann Arbor, MI (US); Kurt A. Dasse, Ann Arbor, MI (US)

(73) Assignee: Cardiac Assist Holdings, LLC, Ann Arbor, MI (US)

(21) Appl. No.: 17/777,110

(22) PCT Filed: Nov. 16, 2020
(86) PCT No.: PCT/US2020/060668

§ 371 Date: May 16, 2022

Related U.S. Application Data
(60) Provisional application No. 62/935,683, filed on Nov. 15, 2019.

(51) Int. Cl. A61M 25/02; A61M 39/02; A61M 1/00; A61F 13/02; A61M 60/88

(52) U.S. Cl. CPC A61M 25/02; A61M 39/0247; A61M 1/912; A61F 13/0216; A61M 1/98; A61M 1/962; A61M 60/88; A61M 2039/0282; A61F 2013/00412

(57) ABSTRACT

Vacuum dressings with a guide tube are provided for implantable medical devices that inhibit infection associated with in-dwelling devices while encouraging healing of the incision around the device. The vacuum dressings mitigate pooling of fluids that harbor bacteria from between the outer diameter of an inserted implantable medical device and the inner diameter of a guide tube and also in the cylindrical gap, between the outer diameter of an inserted implantable medical device and the inner wall of the subcutaneous tunnel, which remains in fluid communication with skin microflora. Implantable medical devices may also illustratively include a variety of catheters, such as venous access, peritoneal dialysis, and other indwelling venous access catheters that require skin penetration; cannulas; Steinman pins; Kirschner wires; and cardiac assist device lines.

### CROSS REFERENCE TO RELATED APPLICATIONS

This application claims priority benefit of U.S. Provisional Application Ser. No. 62/935,683 filed 15 Nov. 2019, the contents of which are hereby incorporated by reference.

### FIELD OF THE INVENTION

The present invention in general relates to medical devices and systems, and in particular to vacuum dressings with a guide tube for implantable medical devices that reduces the complications associated therewith.

### BACKGROUND OF THE INVENTION

Intravenous catheters act as an attachment point for microorganisms, leading to biofilm formation and infection at the site of insertion or along the surface of the device. Infection of the catheter hub and catheter-related bloodstream infections are major complications for patients with indwelling catheters (e.g., Safdar and Maki, Intensive Care Med. 2004 January; 30(1):62-7; Saint et al., Infect Control Hosp Epidemiol. 2000 June; 21(6):375-80).

Prior attempts at controlling catheter-related infection are directed to sterilization techniques such as by topical or fluidic antibacterials applied to the insertion site or integrated into the catheter itself. The antimicrobial lytic activity of C₁-C₈ alcohols is well known. Isopropyl alcohol at a concentration of 60-70% is widely used as an antimicrobial agent for sanitization of surfaces and skin. A concentration of 10% ethyl alcohol inhibits the growth of most microorganisms, while concentrations of 40% and higher are generally considered bactericidal (Sissons et al., Archives of Oral Biology, Vol. 41, 1, JN 1996; 27-34).

Catheters and other in-dwelling medical devices can be kept in place for as little as a few seconds for drainage or delivery of a substance. Besides catheters, other such devices illustratively include cannulas, lines for left ventricular assist devices (LVADs) chest tubes, and the like. It is increasingly common, however, for such devices and specifically peripherally inserted central catheters (PICC), skeletal guide wires, cardiac assist device lines, to be kept in place for weeks, months, or even years. The increased time in which such devices are maintained across the skin increases the likelihood of incision cleft related infection around such devices.

Another common implantable device that breaks the skin and may be a source of infection are blood pumps that may be surgically implanted in, or adjacent to the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly. Typically, the blood pump systems include a driveline that serves as a power and/or signal conduit between the blood pump internal to the patient and a controller/console external to the patient. Additional external medical devices may illustratively include implantable pumps such as insulin pumps and colostomy bags.

Percutaneous access devices (PAD) have been introduced that serve as semi-permanent or extended entry points for the aforementioned catheters and implantable and externally worn medical devices. For example, a percutaneous access device (PAD) may be surgically implanted in the body at the location in the skin where the driveline penetrates the skin to provide a through-the-skin coupling for connecting the supply tube to an extra-corporeal fluid pressure source. In a further example, electrical leads from electrodes implanted in the myocardium are likewise brought out through the skin by means of the PAD. Percutaneous access devices may also illustratively be used for other devices including peritoneal dialysis catheters, Steinman pin, Kirschner wires, and chronic indwelling venous access catheters that require skin penetration. More generally, medical appliances which are implanted so as to cross the skin surface and therefore violate the “barrier function” of the skin, may also illustratively be used for other medical purposes including peritoneal dialysis catheters and, chronic indwelling venous access catheters, neurologic prostheses, osseointegrated prostheses, drug pumps, and other treatments that require skin penetration.

The use of percutaneous access devices inhibits penetration or complications due to the presence of an agent in a subject. As used herein an “agent” is illustratively: an infectious agent such as bacteria, virus, fungus, other organism; or foreign material. Illustrative examples of foreign material include: bandage; soil; water, saliva, urine, or other fluid; feces; chemicals; or other matter known in the art. Illustrative examples of infectious agents that are prevented from penetrating or produce complications include P. aeruginosa, E. cloacae; E. faecalis; C. albicans; K pneumonia; E. coli; S. aureus; or other infectious agents. As used herein, the term “subject” refers to a human or non-human animal, optionally a mammal including a human, non-primate such as cows, pigs, horses, goats, sheep, cats, dogs, avian species and rodents; and a non-human primate such as monkeys, chimpanzees, and apes; and a human, also denoted specifically as a “human subject”.

A draw force is typically applied to a PAD to counteract fluid collection or flow along a percutaneous instrument tissue interface. It is common for fluid to develop in the space surrounding a percutaneous instrument often beginning immediately after insertion. The presence of this fluid allows migration, flow, or other penetration of agents normally excluded by the intact skin to areas below the skin. The penetration by these agents may lead to development of infectious disease, inflammation at the site of insertion, or other unwanted complications. A draw force that is applied is vacuum or hydrodynamic draw through capillary action. A draw force illustratively prevents fluid from moving along an interface between tissue and the embedded catheter or other instrument. The negative pressure of the draw allows the natural pressures of biological material or other atmospheric pressure to move unwanted material away from the areas at or below the site of insertion.

The surface of the driveline, or of the PAD used in cardiac assist systems may have characteristics which promote the formation of a natural biologic seal between the skin and the device to form a barrier to microbial invasion into the body at the skin penetration site. However, a common problem associated with implantation of a percutaneous access device (PAD) is skin regeneration about the periphery of the device to form an immunoprotective seal against infection. New cell growth and maintenance is typically frustrated by the considerable mechanical forces exerted on the interfacial layer of cells. In order to facilitate skin regeneration about the exterior of a PAD, subject cells are often harvested and grown in culture onto PAD surfaces for several days prior to implantation in order to allow an interfacial cell layer to colonize PAD surfaces in advance of implantation. Unfortunately, cell culturing has met with limited acceptance owing to the need for a cell harvesting surgical procedure preceding the implantation procedure. Additionally, maintaining tissue culture integrity is also a complex and time-consuming task.

As an alternative to cell culturing on a percutaneous access device, vacuum assisted wound treatment about a percutaneous access device has been attempted. While DACRON® based random felt meshes have been used to promote cell regrowth in the vicinity of a wound, such felts have uncontrolled pore sizes that can harbor bacterial growth pockets.

U.S. Pat. No. 7,704,225 solves many of these aforementioned problems by providing cell channeling contours, porous biodegradable polymers and the application of vacuum to promote cellular growth towards the surface the neck of a PAD. The facilitating of rapid cellular colonization of a PAD neck allows the subject to act as their own cell culture facility, and as such affords more rapid stabilization of the PAD, and lower incidence of separation and infection. Coating substances thereon illustratively include cell growth scaffolding matrices as detailed in U.S. Pat. Nos. 5,874,500; 6,056,970; and 6,656,496; and Norman et al. Tissue Eng. March 2005, 11(3-4) pp. 375-386.

An outer sleeve of a PAD functions to segregate or deliver vacuum draw pressure to an inner sleeve. The outer sleeve optionally circumferentially and longitudinally covers an inner sleeve. This configuration optionally shields the inner sleeve from epidermal bacterial or other agents upon insertion. An outer sleeve is optionally tapered at one or both ends. Tapering at a distal end (the end nearest the internal end of the catheter during use) provides improved insertion of the instrument into the skin of a subject. A taper may form a smooth interaction with the catheter at the outer sleeve distal end or a ridge is optionally present at or near the site of device interaction with the catheter.

A vacuum source can be any source operable for creating negative pressure in or around the device. A vacuum source is optionally a passive vacuum such as a vacuum tube or bottle, or an active vacuum source illustratively a mechanical pump, a syringe, or other vacuum source. A hydrodynamic draw agent is provided that draws fluid from the tissue surrounding through the sleeve via the conduit. A hydrodynamic draw source illustratively includes a super absorbent polymer such as sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile; high osmotic pressure compositions, such as water soluble salts; and capillary flow draw agents such as dry silica, or other dry hydrophilic powders such cellulosic material. It is noted that recently a diaper filler in the form of a polyacrylic acid as a superabsorbent polymer and a bactericidal skin wash chlorhexidine has been used to draw fluid from the tissue surrounding the sleeve via the conduit. However, this approach to fluid draw has limited and exponentially decaying moisture draw.

FIG. 1 illustrates wearable and implanted components of an exemplary prior art cardiac assist system. A PAD 10 serves as an attachment point for an external supply line 12 that supplies air or fluid from a wearable external drive unit (EDU) 14. The EDU 14 is powered by a wearable battery pack 16. Inside the body of the patient, a drive line 18 is attached to the PAD 10 and provides an air or fluid conduit to a cardiac assist device 20.

FIG. 2 depicts a PAD generally at 100 as shown in U.S. Pat. No. 10,258,784. A cap 102 is formed of a material such as silicone, a polymer or a metal and serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 12d. The inner sleeve 12d is characterized by a large and rigid pore matrix 18 in fluid communication to a vacuum source 22 such that the source 22 draws (arrow 22D) tissue fluid and fibroblasts 21 into the sleeve 12d. Sleeve 12d has a surface 24 that is optionally nanotextured to promote fibroblast adhesion. The surface 24 is optionally decorated with a pattern of contoured cell-conveying channels. It is appreciated that inner sleeve 12d optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcutaneous layer S. A flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

FIGS. 3A-3C illustrate a modular external interface housing 200 coupled to the PAD 100 as disclosed in U.S. Patent Publication US2018/043069. The modular external interface 200 forms a collar about the neck 110 of the PAD 100 with the main body 216 with a locking feature 218, such as a male extension that engages a female receptacle or cavity as a mechanical overlap connection. In a specific embodiment the main body 216 is made of silicone. The collar seal between the main body 216 and the neck 110 of the PAD 100 forms a hermetic seal with a gasket 230, which in a specific embodiment is a flexible gasket integrated into the main body 216. In a specific embodiment the gasket 230 may be a floating gasket. The stabilization of the PAD 100 within the skin to form a germ-free barrier requires subject cells to grow onto the neck surfaces 17 as shown in FIG. 2 of the PAD 100 adjacent to the subject's epidermis E. The neck surface region 17 is adapted to promote growth of autologous fibroblast cells thereon. A suitable exterior side surface substrate for fibroblast growth is a nanotextured polycarbonate (LEXAN®). The modular external interface 200 has a central opening adapted for at least one drive line 220 for insertion into a PAD, and a portal 224 for a vacuum line 222.

The modular external interface 200 is secured and sealed to an outer layer of a patient's skin with a medical dressing. In a specific embodiment the medical dressing is a preform patterned and shaped to conform to the exterior of the modular external interface 200. In a specific embodiment the medical dressing preform may be in two halves (212, 214) that overlap. In a specific embodiment the medical dressing preform may be transparent. In a specific embodiment the medical dressing preform may be made of Tegaderm™ manufactured by Minnesota Mining and Manufacturing Company.

Despite the advances in PAD design and the securement of PAD to a subject's skin there continues to be a problem of disrupting the formation and maintaining of skin layers about the PAD with respect to flexible or pliable drivelines during the healing process. In addition, while vacuum pumps, capillary draw, and hydrodynamic draw have been used reduce the pressure on the insertion site and thereby dry the insertion site to stimulate granulation that will mechanically stabilize the appliance and reduce the prospect of infection; infection at the site of device insertion used with pliable and flexible drivelines continues to occur as the seal between the layers of skin and the bendable driveline tends to either not fully form or fails as the driveline flexes at the insertion site. Furthermore, there continues to be a need to adjust pressure to preclude skin prolapse around a catheter or other temporarily or in-dwelling medical device.

There is a continuing need for improved dressings for percutaneous access devices that encourage and expedite nascent layers of skin that are being formed during the healing process, as well as maintaining an infection preventive seal with evacuation of fluids that support bacteria that lead to infection around percutaneous access devices

### SUMMARY OF THE INVENTION

A vacuum access therapeutic (VAT) is provided to engage a guide tube extending above and out of an epidermal layer. The VAT includes a central dressing adapted to overlay the epidermal layer to surround a guide tube, an outer dressing surrounding the central dressing, and a locating ring intermediate between the central dressing and the outer dressing. A connector coupling the vacuum access therapeutic to a vacuum source, the connector in gaseous communication with the central dressing and the guide tube.

A process is provided for stabilizing an implanted medical device having a guide tube therearound at a situs emerging from patient epidermis. The process includes attaching a vacuum access therapeutic as described above onto the epidermis at the situs, and coupling the vacuum access therapeutic to a vacuum source. Subsequently, a vacuum is applied in a volume between the guide tube and the implanted medical device.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIG. 1 illustrates prior art wearable and implanted components of a cardiac assist system with a percutaneous access device (PAD) and internal driveline;

FIG. 2 is a prior art, partial cutaway view of a flanged percutaneous access device (PAD) with relative dimensions of aspect exaggerated for visual clarity;

FIGS. 3A-3C are perspective views of a prior art modular external interface seal for a PAD appliance secured with adhesive dressings to a subject;

FIG. 4 is a perspective view of a VAT with a guide tube extending above and out of the epidermal layer in accordance with embodiments of the invention;

FIG. 5A is a cut away view of the VAT depicted in FIG. 4 attached to a subject with a cross-sectional view of a guide tube with a fibroblast adhesion feature in the dermal layer in accordance with embodiments of the invention;

FIG. 5B is a cut away view of the VAT depicted in FIG. 4 attached to a subject with a cross-sectional view of a guide tube without the fibroblast adhesion feature in the dermal layer in accordance with embodiments of the invention;

FIG. 6A is a top view of a VAT that has a central dressing with a segmented orifice prior to insertion of a catheter in accordance with embodiments of the invention;

FIG. 6B is a side view of the VAT of FIG. 6A with a catheter inserted in the central dressing with the segmented orifice that shows the segments extending downward into the dermal layers of a subject in accordance with embodiments of the invention;

FIGS. 7A-7F are a series of views that illustrate the deployment of a central dressing for application on the epidermal surface of a subject in accordance with embodiments of the invention;

FIG. 8A is a perspective view of the VAT of FIG. 4 that illustrates the outer dressing in accordance with embodiments of the invention;

FIG. 8B is a rear side view that illustrates the VAT of FIG. 4 that illustrates the connecting ports in accordance with embodiments of the invention;

FIG. 8C is a perspective view of the VAT of FIG. 4 that illustrates the outer dressing and a catheter inserted through the central dressing in accordance with embodiments of the invention;

FIG. 9 is a fully assembled VAT with the manual pump of FIG. 13 in accordance with embodiments of the invention;

FIG. 10 is a perspective view of a foam support ring positioned about the central ring of the VAT of FIG. 9 in accordance with embodiments of the invention;

FIG. 11 is a perspective view of a central ring and central dressing of the VAT of FIG. 9 prior to deployment on a subject in accordance with embodiments of the invention;

FIGS. 12A and 12B are perspective views of a clip for use with embodiments of the VAT in accordance with embodiments of the invention;

FIG. 13 is a perspective view of the VAT of FIG. 9 deployed on a subject with the foam support ring and clip of FIG. 11 applied in accordance with embodiments of the invention;

FIGS. 14A-14C are a series of views of a manual pump for use with the VAT of FIG. 9, where FIG. 14A is a perspective view, FIG. 14B is an underside perspective view; and FIG. 14C is a cutaway view, respectively;

FIG. 15 is a side cut away view of a catheter inserted in a guide tube with a spiral thread inserted in the gap between the catheter and the inner wall of the guide tube in accordance with embodiments of the invention; and

FIG. 16 is a side cut away view of a catheter inserted in a guide tube with a set of threads inserted in the gap between the catheter and the inner wall of the guide tube in accordance with embodiments of the invention.

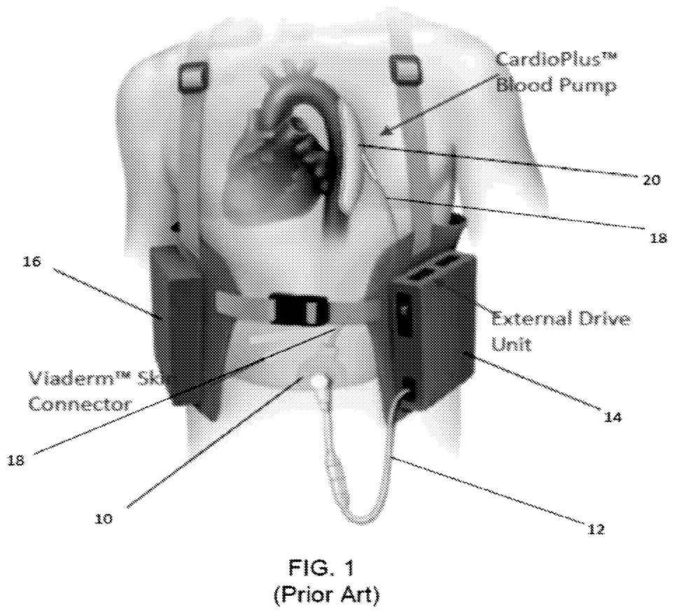

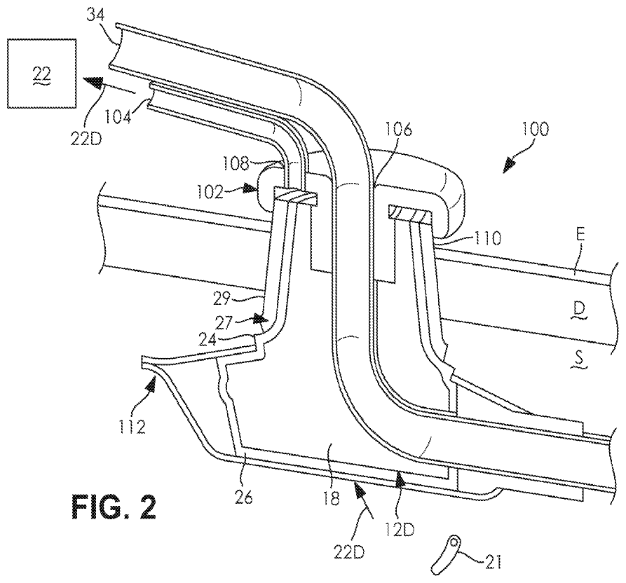

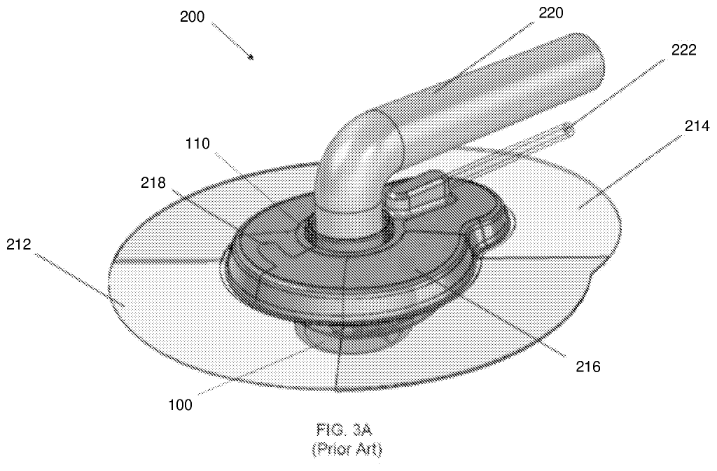

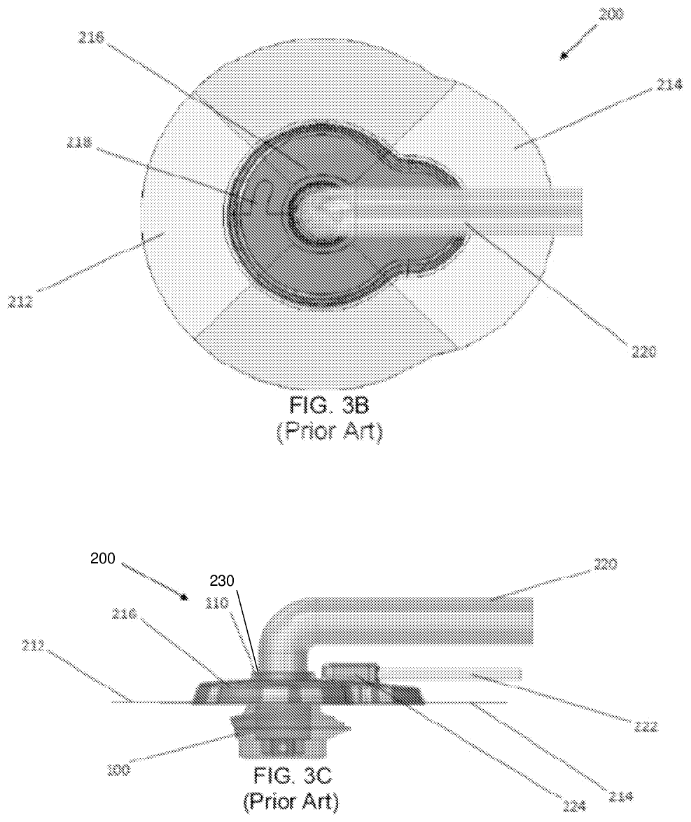

### DETAILED DESCRIPTION OF THE INVENTION

Embodiments of the invention provide vacuum dressings with a guide tube for implantable medical devices that inhibits infection and in some inventive embodiments associated with in-dwelling devices encourages healing of the incision around the device. Inventive vacuum dressings mitigate pooling of fluids that harbor bacteria from between the outer diameter of an inserted implantable medical device and the inner diameter of a guide tube and also in the cylindrical gap, between the outer diameter of an inserted implantable medical device and the inner wall of the subcutaneous tunnel, which remains in fluid communication with skin microflora. Implantable medical devices may also illustratively include a variety of catheters, such as venous access, peritoneal dialysis, and other indwelling venous access catheters that require skin penetration; cannulas; Steinman pins; Kirschner wires; and cardiac assist device lines.

Previous efforts have concentrated on complete removal of moisture or humidity from wound areas, however, according to the present invention, it is noted that in some instances, a level of moisture is required to allow fibroblasts to actively attach to an implanted device or tube, or to a localized fibroblast attachment feature, surrounding such a device and to promote the establishment of intact biological barrier function of the stratum corneum layer of skin. According to the present invention, it is also noted that moisture and pressure levels may be needed to change as the wound healing process progresses through different stages. According to the present invention, it is further noted that pressure levels may require adjustment to preclude skin prolapse around an implanted device. In specific inventive embodiments, a vacuum applies an intermittent negative pressure or oscillating negative pressure or negative pressure modulated based on real-time measurements of relevant physiologic parameters such as tissue oxygenation, local pH, moisture egressing from the subcutaneous tunnel, and the like. This has been found to be surprisingly effective in preventing tissue prolapse, telangiectasias, and sealing of distal subcutaneous pockets that can harbor fluids and microbes. The magnitude of the negative pressure is optionally adjustable, constant, or variable. Intermittent vacuum can be applied for periods of 1 millisecond to 12 hours with rest periods therebetween that independently vary from 1 millisecond to 12 hours. The rest periods are either a venting to ambient room pressure or a cessation of active vacuum draw to allow a slow increase in pressure towards ambient pressure through gas or fluid leakage into the central dressing. It is appreciated that a comparatively weak vacuum draw pulsed rapidly on and off on the order of 1 millisecond to 10 seconds simulates the behavior of hydrodynamic draw without the need for routine monitoring and changing of materials. Embodiments of the inventive guide tube are intended to be changed every 1 to 7 days as needed. Embodiments of the inventive guide tube have the following characteristics:

i) Multiple channels, grooves, and/or open cell interconnected channels capable of conducting vacuum from the exterior vacuum dressing, along the length of the subcutaneous tunnel, to remain in fluid communication with a fibroblast adhesion feature.

ii) Material properties such that the multiple channels identified in i) above resist collapse under the influence of the vacuum applied to the system.

iii) Material properties such that, during the act of removal of the guide tube, the external surface of the guide tube is non-adherent and resists adhesive interference/abrasion with epidermal cells which have migrated from the external epidermis along the walls of the subcutaneous tunnel. This process of epidermal downward migration into the subcutaneous tunnel is referred to as “Marsupialization”.

iv) Geometric properties such that, in the act of removal of the guide tube (“peel away functionality”), heavy disruptive forces delivered to the healing fibroblasts and epidermal cells are minimized Such geometries may include, but are not limited to, pre-formed perforations, pull-strips, circumferential segmentation into multiple parallel or non-parallel longitudinal ribbon-like or thread-like segments which anticipate the “peel-away” functionality, or a spiral configuration allowing a spiral-on application procedure and a spiral-off removal procedure. The segments of the guide tube can optionally include a stiffening element.

v) Material and geometric properties of the guide tube which are compatible with an insertion tool which aid in placement of the vacuum guide sleeve within the gap space between the walls of the subcutaneous tunnel and the therapeutic catheter.

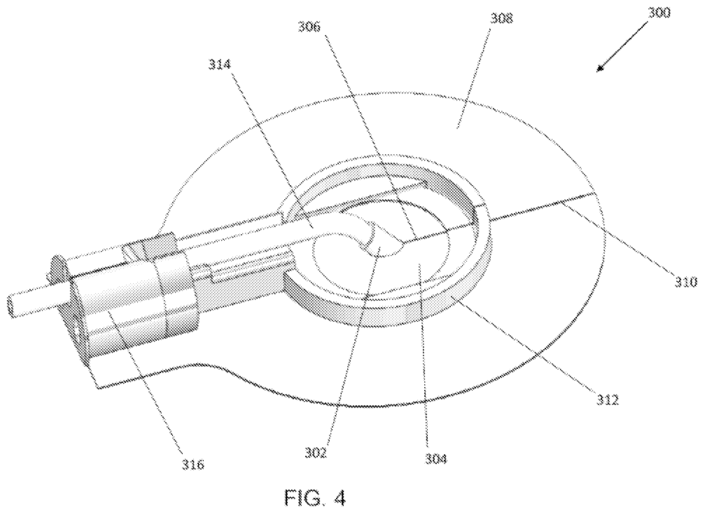

Referring now to the figures, FIG. 4 is a perspective view of an inventive vacuum access therapeutic (VAT) 300 with a guide tube 302 extending above and out of the epidermal layer E and through a central dressing 304 with a dividing slit 306. While the central dressing 304 is depicted as being circular in shape, it is appreciated that the central dressing is also readily formed as a triangle, rectilinear, pentagonal, hexagonal, other polygonal shapes, as well as irregular shapes. The section of the guide tube 302 extending above the central dressing 304 is in fluid communication with the vacuum source applied to the dressing. The guide tube 302 may be made of materials so as to resist adhesion to the walls of the subcutaneous tunnel. Perfluoropolymers and polyethylene terephthalate (PET) are exemplary of such materials. An outer dressing 308 with a slit 310 surrounds the central dressing 304 and a locating ring 312. A connector 316 has a slot and port for an implantable medical devices, depicted throughout and referred to generically as a catheter, 314. The inner central dressing 304 and outer dressing 308 may be formed of a medical grade clear polymer such as an acrylic polymer, a polycarbonate, PET, Polyether ether ketone (PEEK), or combinations thereof. Other suitable dressings illustratively include the Tegaderm™ (3M) BioPatch™ (Medtronic) with chlorhexidine-based dressings that are opaque or transparent, and Prevahex™ (Enterotech Life Sciences). The underside of the inner central dressing 304 and the outer dressing 308 have a peelable adhesive applied that sticks to the skin of a subject.

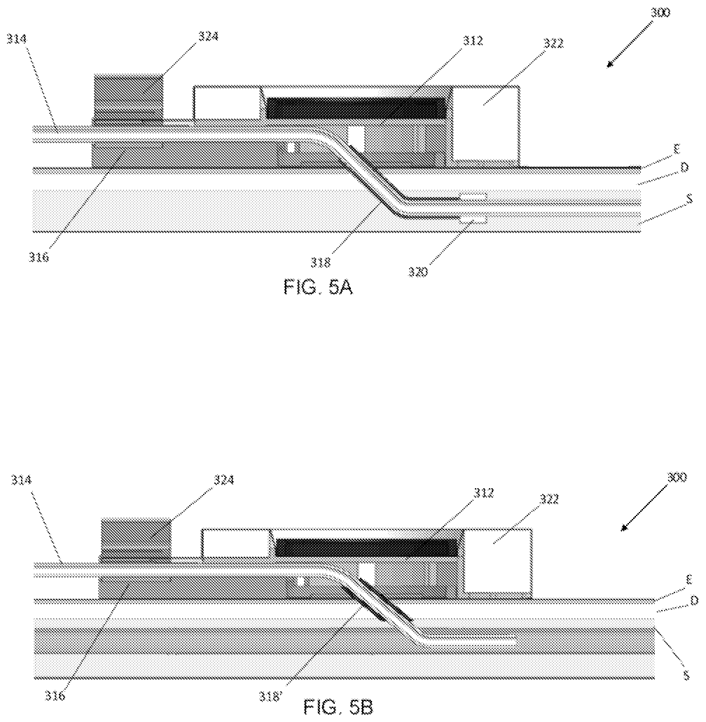

FIG. 5A is a cut away view of the VAT 300 attached to a subject with a cross-sectional view of a guide tube 318 with a fibroblast adhesion feature 320 in the subcutaneous tissue layer S. The fibroblast adhesion feature 320 may be formed from materials as described in U.S. Pat. No. 10,065,030 which is included herein in its entirety by reference. Illustrative materials include collagen, PEBAX, nylons, polypropylenes, polyurethanes, polyethylenes (HDPE, UHWPE, LDPE, or any blend of the aforementioned polyethylenes), PET, NiTi, MYLAR, Nickel Titanium Alloy, other polymers such as perfluoropolymers, other thermoplastic polymers, fabrics, silicones such as silicone rubber, latex, glass, or other materials known in the art. It is appreciated that polymeric materials with a gradient of cross-linking density through the material afford certain advantages with respect to promoting vacuum or hydrodynamic draw and fibroblast infiltration. In a specific inventive embodiment, the fibroblast adhesion feature 320 may be a cuff. In a specific inventive embodiment fibroblast adhesion feature 320 may be made of dacron which under the action of an applied vacuum reduces bioburden within the access wound and between the inner surface of the guide tube 318 and the outer surface of the implantable medical devices such as the catheter 314 shown. An airtight cover 322 encloses the area defined by the locating ring 312 and contains a vacuum above the entry point of the implantable medical devices.

When a fibroblast adhesion feature is available (e.g., Dacron Cuff, nano textured polycarbonate PAD, expanded perfluoropolymers, etc.), the role of the non-adhering guidance tube during the initial post-implant wound healing phase is to:

a) Provide fluid communication of negative pressure wound therapy to the gap between the therapeutic catheter and the wall of the subcutaneous tunnel so as to reduce bioburden accumulation within the subcutaneous tunnel.

b) Provide fluid communication of negative pressure wound therapy to the Fibroblast adhesion feature so as to reduce bioburden associated with the fibroblast adhesion feature and to encourage fibroblast infiltration and adhesion of the fibroblast adhesion feature.

c) During dressing changes that include changing of the non-adhering guidance tube, provide for gentle mechanical debridement of the gap between the therapeutic catheter and the wall of the subcutaneous tunnel while avoiding injury to migrating epithelial cells as they down migrate along the walls of the subcutaneous tunnel to reach the fibroblast adhesion feature (a process referred to as “marsupialization”).

When a fibroblast adhesion feature (e.g., Dacron cuff, nano textured polycarbonate PAD, etc) is available, the role of the non-adhering vacuum guidance tube during the post-implant maintenance phase is to:

a) Provide fluid communication of negative pressure wound therapy to the gap between the therapeutic catheter and the wall of the subcutaneous tunnel so as to reduce bioburden accumulation within the subcutaneous tunnel.

b) During dressing changes that include changing of the non-adhering guidance tube, provide for gentle mechanical debridement of the gap between the therapeutic catheter and the wall of the subcutaneous tunnel while avoiding injury to migrating epithelial cells as they down migrate along the walls of the subcutaneous tunnel to reach the fibroblast adhesion feature (a process referred to as “marsupialization”)

FIG. 5B is a cut away view of the VAT 300 depicted in FIG. 4 attached to a subject with a cross-sectional view of a guide tube 318′ without the fibroblast adhesion feature in the dermal layer. Examples of such a VAT lacking a fibroblast adhesion feature are commonly used in the context of a medical devices that illustratively include short-term intravenous catheters, short term intraarterial catheters, Steinman pins, and the like. In the absence of a fibroblast adhesion feature, the role of the negative-pressure wound therapy of an applied vacuum is to reduce bioburden accumulating in the gap between the outerwall of the medical device and the walls of the subcutaneous tunnel.

When a fibroblast adhesion feature (e.g. Dacron cuff, nano textured polycarbonate PAD, etc) is not integrated into the therapeutic catheter (as would be the case with IV catheters, intraarterial catheters, Steinman pins, etc., the role of the short non-adhering Vacuum guidance tube during the post-implant phase is to provide fluid communication of negative pressure wound therapy to create a hydrostatic gradient to the gap between the therapeutic catheter and the wall of the subcutaneous tunnel so as to reduce bioburden accumulation within the subcutaneous tunnel.

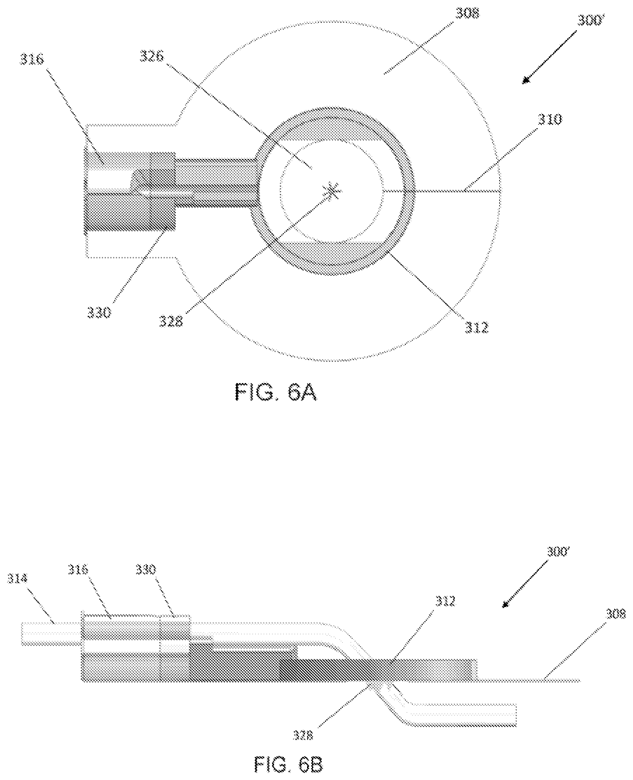

FIG. 6A is a top view of a VAT 300′ that has a central dressing 326 with a segmented orifice 328 prior to insertion of an implantable medical device. The segments 328 of the orifice flex downward through the dermis (may be aided with a spiral slide tool) and into the entry point or wound. The number of segments may vary from 5-20. The segments have no adhesive on their undersides. The segments or petals 328 permit vacuum passage into the catheter puncture wound. FIG. 6A has no lateral slit (such as 306 in FIG. 4) and requires the catheter to be advanced after the catheter dressing is secured to the skin. The catheter is placed from outside the body to inside the body. If a lateral slit is present in the central dressing 326, the central dressing 326 may be placed about the catheter if the catheter is placed from inside the body to outside, or placed outside the body to inside the body with the dressing placed afterward. FIG. 6B is a side view of the VAT 300′ of FIG. 6A with a catheter 314 inserted in the central dressing 326 with the segmented orifice 328 that shows the segments or petals 328 extending downward into the dermal layers of a subject to direct vacuum into the wound created for insertion of the medical device.

It is appreciated that as a therapeutic catheter 314 is passed through the segmented orifice 328 opening, the petals will bend from the plane of the epidermis to follow the outer wall of the therapeutic catheter. The portions of the petals remaining undisturbed in the plane of the epidermis will remain in immediate adjacency. However, the portions of the petals which have bent to follow the outer wall of the therapeutic catheter 314 will spread apart from their adjacent neighboring petals. This spreading will allow fluid communication of the vacuum to portions of the wall of the subcutaneous tunnel. The inventive alternate pattern will allow precise accommodation of the central dressing 326 to variations in the outer diameter of the therapeutic catheter 314.

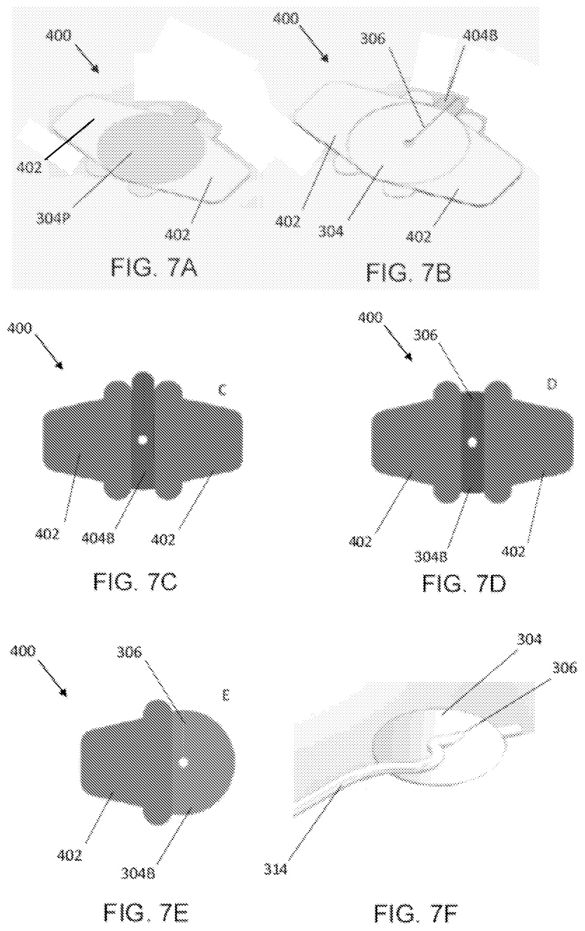

FIGS. 7A-7F are a series of views that illustrate the deployment of a central dressing 304 for application on the epidermal surface of a subject. A fully packaged central disc dressing 400 is shown in FIG. 7A with protective tab sheets 402 and 304P pre-applied. In FIG. 7B protective sheet 304P is removed to expose the top side of the central dressing 304 with the slit and center hole. FIG. 7C is a bottom view of the packaged central disc dressing 400 with the bottom center tab 404B. In FIG. 7D the bottom center tab 404B is removed, which allows the user to pull on outer protective tab sheets 402 to spread the slit 306 apart for placement of the central dressing 304 about an implanted medical device such as a catheter. The bottom of the central dressing 304 has an adhesive to tack the center dressing to the skin of a subject. In FIG. 7E the outer protective tab sheets 402 are removed to fully expose the adhesive on the bottom of the central dressing 304 for attachment to the subject. In FIG. 7F a catheter 306 is positioned in the deployed central dressing 304.

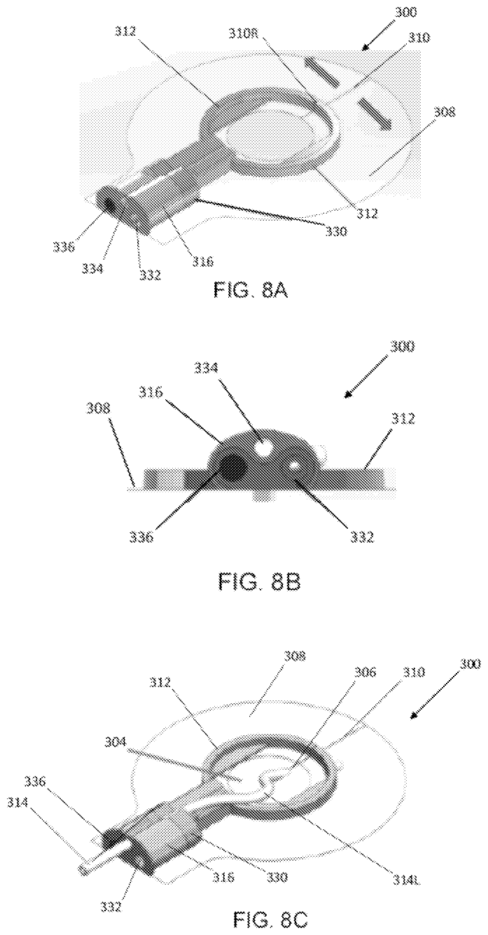

FIG. 8A is a perspective view of the VAT 300 of FIG. 4 that illustrates the outer dressing 308 with a slit 310 that surrounds the central dressing 304. In addition, the locating ring 312 also has a slit 310R. As indicated by the arrows, in use the VAT 300 is spread about the split line 310 and placed about the medical appliance or catheter. The locator ring 312 is centered about catheter that is the emerging from the central dressing 304. The catheter is pressed into the slot of the stabilizer 330 and connector 316. As shown in FIG. 8C, some slack 314L is left in the catheter 314 to permit sliding and deflection as a subject bends and twists. FIG. 8B is a rear side view of the VAT 300 that illustrates the connecting ports including, a vacuum port 332, a medical appliance or catheter port 334, and a one way valve or “slow leak” port 336. FIG. 8C is a perspective view of the VAT 300 that illustrates the outer dressing 308 and a catheter 314 inserted through the central dressing 304.

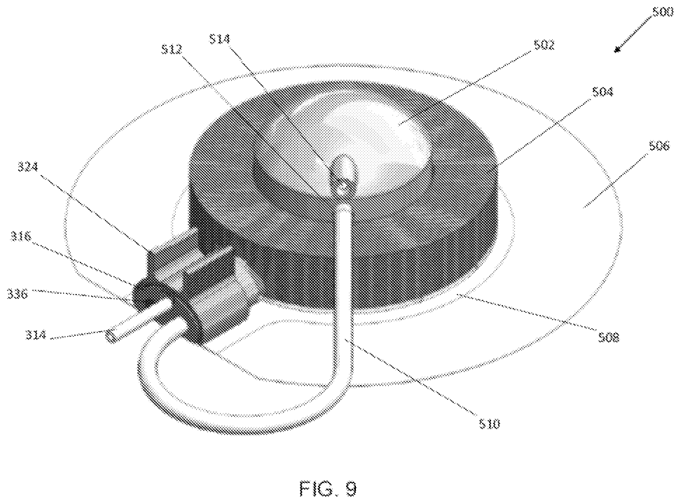

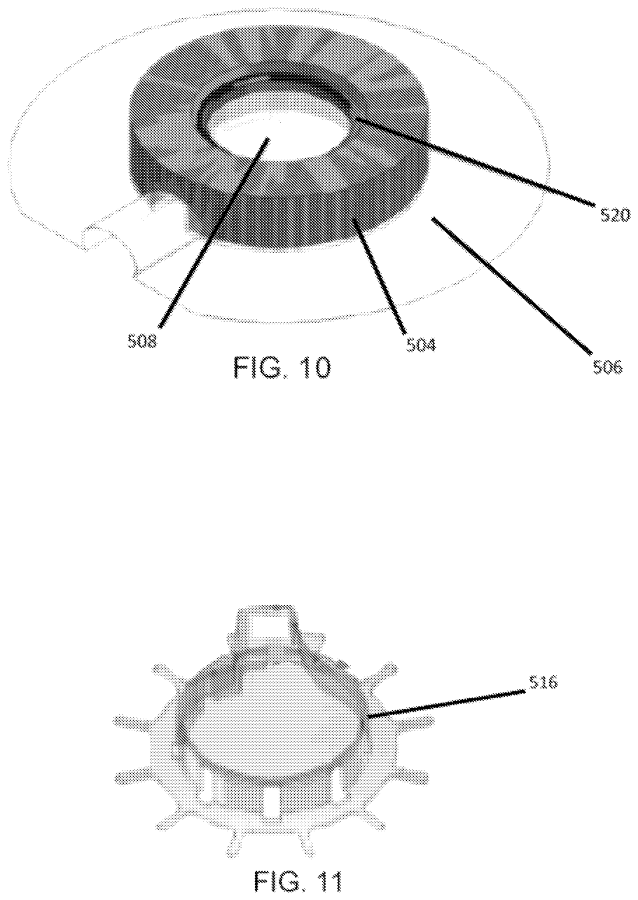

FIG. 9 is an inventive embodiment of a fully assembled VAT 502 with a manual pump actuated by compressing a flexible diaphragm 502. The diaphragm rests on top of a foam support ring 504. An outer dressing 506 surrounds an inner central dressing 508. A vacuum line 510 is connected to the vacuum port 512 on the flexible diaphragm 502 and extends to connect to the vacuum port 332 in the connector 316. A clip 324 seals the outer dressing 506 to the connector 316. The outer dressing 506 may be a urethane thermoform skin with peripheral skin adhesive. A one-way valve 514 releases air when the diaphragm 502 is depressed to create the vacuum when the diaphragm 502 expands after being pressed. The diaphragm acts in a similar manner to a primer button. FIG. 10 is a perspective view of the foam support ring 504 positioned about the central ring 516 of the VAT 500. Also visible in FIG. 10 is a pump friction ring 520 that retains the flexible diaphragm 502 with a press fit. FIG. 11 shows the central ring 516 and central dressing 508 with backing release paper still applied prior to deployment on a subject.

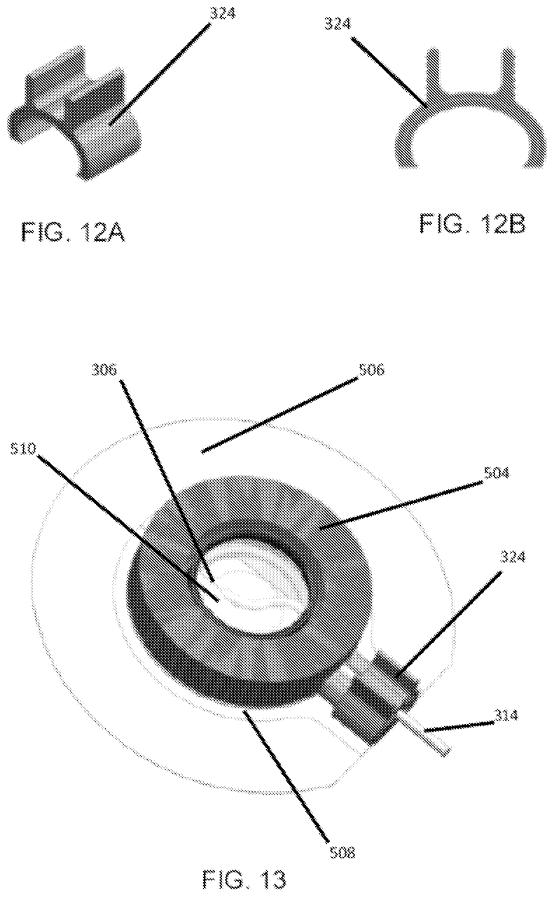

FIGS. 12A and 12B are perspective views of a clip 324 for use with embodiments of the VAT (300, 300′, 500). Upon placement of the vacuum dressing assembly over the skin dressing, the clip 324 is applied to seal the system. The clip 324 has an inward spring bias.

FIG. 13 is a perspective view of the VAT 500 deployed on a subject with the foam support ring and clip of FIG. 12 applied in accordance with embodiments of the invention;

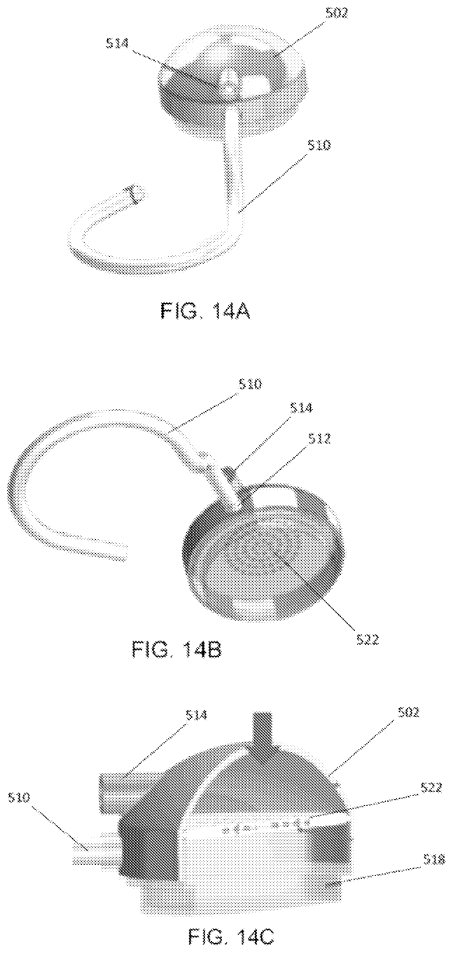

FIGS. 14A-14C are a series of views of a manual pump for use with the VAT of FIG. 9, where FIG. 14A is a perspective view, FIG. 14B is an underside perspective view; and FIG. 14C is a cutaway view, respectively. As seen in FIGS. 14B and 14C a filter 522 is supported in the volume below the diaphragm 502. In a specific inventive embodiment, the filter 522 is a 0.22μ hydrophobic filter. An exudate collection vessel 518 collects the exudate. In a specific embodiment the volume of the collection vessel 518 is approximately 25 ml. It is appreciated that an electronic pump and controller may be substituted for the manual pump.

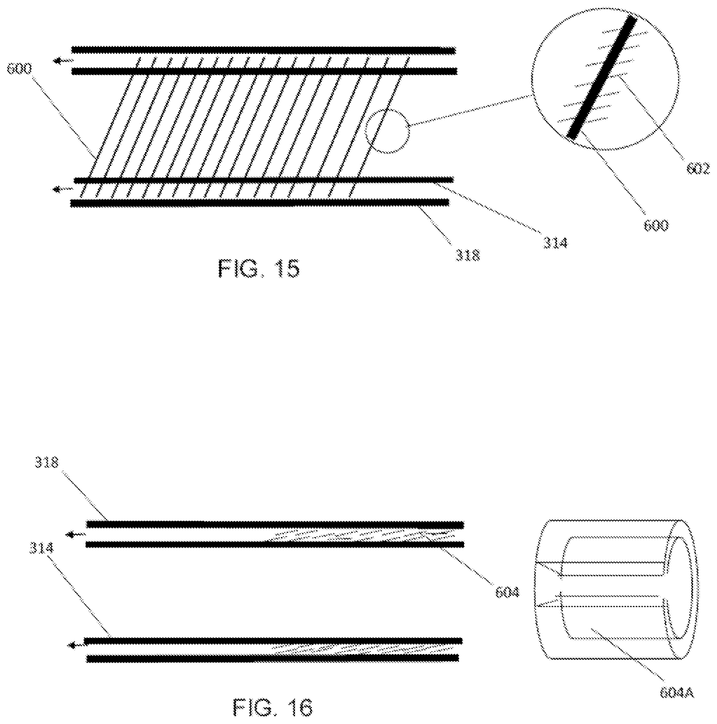

FIG. 15 is a side cut away view of a catheter 314 inserted in a guide tube 318 with a spiral thread 600 inserted in the gap between the catheter 314 and the inner wall of the guide tube 318. The spiral thread may have smaller fibers 602 emanating from the main body of the spiral thread 600. A vacuum as signified by the arrows pulls or wicks bacteria causing fluids up and out from between the interface of the catheter 314 and the inner wall of the guide tube 318. The spiral thread also supports the walls of the guide tube 318 that are under pressure from the vacuum. It is appreciated that the vacuum may be pulsed to lessen the pressure on the guide walls.

FIG. 16 is a side cut away view of a catheter 314 inserted in a guide tube 318 with a delimiter 604 therebetween and contacting the exterior of the catheter 314 and interior of the guide tube 318. The delimiter 604 is shown as a spiral inserted in the gap between the catheter 314 and the inner wall of the guide tube 318. The delimiter 604 functions to preclude collapse of the guide tube 318 against the implanted medical device under the effects of vacuum thereby facilitating fluid draw under conditions that would otherwise isolate distal pockets of fluid from vacuum absent the delimiter 604. While the delimiter 604 is depicted as a spiral, it is appreciated that linear strands and a cylinder with a sidewall split as shown at 604A are also suitable configurations for a delimiter. In some inventive embodiments, the delimiter 604 is tufted to promote wicking of fluid from implant situs. It is further appreciated that a delimiter 604A also serves as an installation appliance to place a delimiter 604 intermediate between the catheter 314 and the guide tube 318. A vacuum as signified by the arrows pulls or wicks bacteria causing fluids up and out from between the interface of the catheter 314 and the inner wall of the guide tube 318 via the threads 604. The threads also support the walls of the guide tube 318 that are under pressure from the vacuum. It is appreciated that the vacuum may be pulsed to lessen the pressure on the guide walls.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A vacuum access therapeutic adapted to engage a guide tube extending above and out of an epidermal layer comprising:
a central dressing adapted to overlay the epidermal layer to surround the guide tube;
an outer dressing surrounding the central dressing;
a connector coupling the vacuum access therapeutic to a vacuum source, the connector in gaseous communication with the central dressing and the guide tube.

2. The vacuum access therapeutic of claim 1 wherein the central dressing is circular.

3. The vacuum access therapeutic of claim 1 wherein the central dressing further comprises a dividing slit adapted to surround the guide tube.

4. The vacuum access therapeutic of claim 1 wherein the central dressing further comprises a segmented orifice adapted to surround the guide tube.

5. The vacuum access therapeutic of claim 1 wherein the connector has a slot or a port adapted to receive an implantable medical device.

6. The vacuum access therapeutic of claim 5 wherein the implantable medical device is a catheter.

7. The vacuum access therapeutic of claim 1 wherein at least one of the central dressing and the outer dressing is optically transparent to an unaided normal human eye.

8. The vacuum access therapeutic of claim 1 further comprising an adhesive layer on an underside of the central dressing, the outer dressing, or both the central dressing and the outer dressing.

9. The vacuum access therapeutic of claim 1 further comprising a protective tab sheet overlying the central dressing.

10. The vacuum access therapeutic of claim 1 further comprising a locating ring intermediate between the central dressing and the outer dressing.

11. The vacuum access therapeutic of claim 1 further comprising a stabilizer exterior to the central dressing.

12. The vacuum access therapeutic of claim 1 wherein the connector further comprises a vacuum port.

13. The vacuum access therapeutic of claim 1 wherein the connector further comprises a one way valve.

14. (canceled)

15. (canceled)

16. The vacuum access therapeutic of claim 1 further comprising a clip adapted to engage an outer portion of the connector.

17. (canceled)

18. The vacuum access therapeutic of claim 1 further comprising a support ring formed of a foamed polymer.

19. (canceled)

20. The vacuum access therapeutic of claim 1 further comprising an exudate collection vessel.

21-23. (canceled)

24. A process of stabilizing an implanted medical device having a guide tube therearound at a situs emerging from patient epidermis comprising:
attaching a vacuum access therapeutic according to claim 1 onto the epidermis at the situs;
coupling the vacuum access therapeutic to a vacuum source; and
applying a vacuum in a volume between the guide tube and the implanted medical device.

25. The process of claim 24 wherein the vacuum is applied intermittently.

26. The process of claim 25 wherein the vacuum is applied with rest periods between application of the vacuum.

27-29. (canceled)
